(19) United States
(12) Patent Application Publication
(10) Pub. No.: US 2010/0292217 A1
(43) Pub. Date: Nov. 18, 2010

(54) RANOLAZINE FOR THE TREATMENT OF CNS DISORDERS

(75) Inventors: Luiz Belardinelli, Palo Alto, CA (US); Alfred George, Brentwood, TN (US); Kristopher Kahlig, Nashville, TN (US); Sridharan Rajamani, Fremont, CA (US)

Correspondence Address: CV THERAPEUTICS, INC.;Gilead Palo Alto, Inc., 333 Lakeside Drive, Foster City, CA, 94404, US

(73) Assignee: Gilead Palo Alto, Inc., Foster City, CA (US)

(21) Appl. No.: 12/779,753

(22) Filed: May 13, 2010

Related U.S. Application Data
(60) Provisional application No. 61/178,170, filed on May 14, 2009.
(60) Provisional application No. 61/279,395, filed on Oct. 20, 2009.

(51) Int. Cl. A61K 31/55; A61K 31/495; A61K 31/53; A61K 31/5513; A61P 25/08; A61P 25/06; A61P 25/00

(52) U.S. Cl. 514/217; 514/252.12; 514/242; 514/221

(57) ABSTRACT

The present invention relates to a method for CNS disorders such as epilepsy and migraine comprising the administration of a therapeutically effective amount of ranolazine.

### CROSS-REFERENCE TO RELATED APPLICATIONS

This application claims priority to U.S. Provisional Patent Application Ser. No. 61/178,170, filed May 14, 2009, and U.S. Provisional Patent Application Ser. No. 61/279,395, filed Oct. 20, 2009, the entire disclosures of which are hereby incorporated by reference.

### FIELD OF THE INVENTION

The present invention relates to method of treating epilepsy and other central nervous system (CNS) disorders by the administration of ranolazine. The method finds utility in the treatment of any CNS condition wherein the inhibition of sodium channels would be beneficial such as epilepsy and migraine. This invention also relates to pharmaceutical formulations that are suitable for such combined administration.

### DESCRIPTION OF THE ART

U.S. Pat. No. 4,567,264, the specification of which is incorporated herein by reference in its entirety, discloses Ranolazine, (±)-N-(2,6-dimethylphenyl)-4-[2-hydroxy-3-(2-methoxyphenoxy)-propyl]-1-piperazineacetamide, and its pharmaceutically acceptable salts, and their use in the treatment of cardiovascular diseases, including arrhythmias, variant and exercise-induced angina, and myocardial infarction. In its dihydrochloride salt form, Ranolazine is represented by the formula:

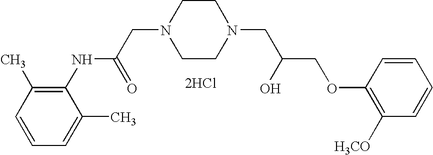

This patent also discloses intravenous (IV) formulations of dihydrochloride Ranolazine further comprising propylene glycol, polyethylene glycol 400, Tween 80 and 0.9% saline.

U.S. Pat. No. 5,506,229, which is incorporated herein by reference in its entirety, discloses the use of Ranolazine and its pharmaceutically acceptable salts and esters for the treatment of tissues experiencing a physical or chemical insult, including cardioplegia, hypoxic or reperfusion injury to cardiac or skeletal muscle or brain tissue, and for use in transplants. Oral and parenteral formulations are disclosed, including controlled release formulations. In particular, Example 7D of U.S. Pat. No. 5,506,229 describes a controlled release formulation in capsule form comprising microspheres of Ranolazine and microcrystalline cellulose coated with release controlling polymers. This patent also discloses IV Ranolazine formulations which at the low end comprise 5 mg Ranolazine per milliliter of an IV solution containing about 5% by weight dextrose. And at the high end, there is disclosed an IV solution containing 200 mg Ranolazine per milliliter of an IV solution containing about 4% by weight dextrose.

The presently preferred route of administration for Ranolazine and its pharmaceutically acceptable salts and esters is oral. A typical oral dosage form is a compressed tablet, a hard gelatin capsule filled with a powder mix or granulate, or a soft gelatin capsule (softgel) filled with a solution or suspension. U.S. Pat. No. 5,472,707, the specification of which is incorporated herein by reference in its entirety, discloses a high-dose oral formulation employing supercooled liquid Ranolazine as a fill solution for a hard gelatin capsule or softgel.

U.S. Pat. No. 6,503,911, the specification of which is incorporated herein by reference in its entirety, discloses sustained release formulations that overcome the problem of affording a satisfactory plasma level of Ranolazine while the formulation travels through both an acidic environment in the stomach and a more basic environment through the intestine, and has proven to be very effective in providing the plasma levels that are necessary for the treatment of angina and other cardiovascular diseases.

U.S. Pat. No. 6,852,724, the specification of which is incorporated herein by reference in its entirety, discloses methods of treating cardiovascular diseases, including arrhythmias variant and exercise-induced angina and myocardial infarction.

U.S. Patent Application Publication Number 2006/0177502, the specification of which is incorporated herein by reference in its entirety, discloses oral sustained release dosage forms in which the Ranolazine is present in 35-50%, preferably 40-45% Ranolazine. In one embodiment the Ranolazine sustained release formulations of the invention include a pH dependent binder; a pH independent binder; and one or more pharmaceutically acceptable excipients. Suitable pH dependent binders include, but are not limited to, a methacrylic acid copolymer, for example Eudragit® (Eudragit® L100-55, pseudolatex of Eudragit® L100-55, and the like) partially neutralized with a strong base, for example, sodium hydroxide, potassium hydroxide, or ammonium hydroxide, in a quantity sufficient to neutralize the methacrylic acid copolymer to an extent of about 1-20%, for example about 3-6%. Suitable pH independent binders include, but are not limited to, hydroxypropylmethylcellulose (HPMC), for example Methocel® EI0M Premium CR grade HPMC or Methocel® E4M Premium HPMC. Suitable pharmaceutically acceptable excipients include magnesium stearate and microcrystalline cellulose (Avicel® pH101).

### BACKGROUND OF THE INVENTION

According to the National Society for Epilepsy there are over 40 different types of epilepsy. Each type is defined by its unique combination of seizure type, age of onset, EEG findings. Location and/or distribution of the seizures are also used to group types of epilepsy. The specific causation of any one type of epilepsy may not be known but it is now known that mutations in the gene SCN1A result in several specific types of epilepsy and CNS disorders.

SCN1A encodes the pore forming α-subunit of the brain voltage-gated sodium (Naᵥ) channel Naᵥ1.1 and is the most commonly mutated gene causing inherited epilepsy. Mutant Naᵥ1.1 channels cause a wide range of epilepsy syndromes from the relatively benign generalized epilepsy with febrile seizures plus (GEFS+) to the debilitating severe myoclonic epilepsy of infancy (SMEI). More recently, mutation of SCN1A has been found to cause the inherited migraine syndrome familial hemiplegic migraine type 3 (FHM3). A common feature observed for several Naᵥ1.1 mutants is a significantly increased persistent current, which is believed to cause neuronal hyperexcitability by facilitating action potential generation and propagation.

Although ranolazine exhibits activity against several molecular targets, the primary therapeutic mechanism of action is thought to be the block of Naᵥ channel persistent current. This effect was first shown in a guinea pig ventricular myocyte model of long QT syndrome (LQT) in which persistent sodium current was induced by the toxin ATX-II (Wu et al. (2004). J Pharmacol Exp Ther 310:599-605; Song et al. (2004). J Cardiovasc Pharmacol 44:192-199. Subsequently, ranolazine was shown to preferentially block the increased persistent current directly carried by Naᵥ1.5 LQT mutant channels (Fredj et al. (2006). Br J Pharmacol 148:16-24; Rajamani et al. (2009). Heart Rhythm 6:1625-1631). More recently, ranolazine has been shown to block various wild-type Naᵥ channel isoforms expressed in muscle (Naᵥ1.4) (Wang et al. (2008). Mol Pharmacol 73:940-948), heart (Naᵥ1.5) (Wang et al, 2008) and peripheral nerves (Naᵥ1.7 and Naᵥ1.8) (Wang, 2008; Rajamani et al. (2008a). Channels 2:449-460).

However, the ability of ranolazine to inhibit brain Naᵥ channel isoforms (such as Naᵥ1.1 or Naᵥ1.2) has not previously been reported. It has now been discovered that ranolazine has the ability to preferentially block the persistent current generated by mutant Naᵥ1.1 channels. Ranolazine exhibits a high affinity inhibition of Naᵥ1.1 in both tonic and use dependent block paradigms. Clinical availability of a Naᵥ1.1 persistent current selective drug such as ranolazine provide a new treatment option for CNS disorders such as SCN1A associated epilepsy and migraine syndromes.

### SUMMARY OF THE INVENTION

The object of the invention is to provide methods for the treatment of CNS disorders, including but not limited to migraine and epilepsy comprising the step of administering to a patient in need thereof a therapeutically effective amount, or a prophylactically effective amount, of Ranolazine, or a pharmaceutically acceptable salt thereof.

In some aspects of the invention, Ranolazine is administered for the treatment or prevention of CNS disorder associated with SCN1A mutation. Conditions associated with mutations in the SCN1A include, but are not limited to, generalized epilepsy with febrile seizures plus (GEFS+) type 2, severe myoclonic epilepsy of infancy (SMEI), familial hemiplegic migraine type 3 (FHM3), generalized epilepsy with febrile seizures plus (GEFS+) type 1.

### SUMMARY OF THE FIGURES

FIG. 1 presents the effect of ranolazine on WT-Naᵥ1.1. FIG. 1(A) shows representative whole-cell sodium currents recorded during sequential superfusion of control solution followed by 30 μM ranolazine. Currents were activated by voltage steps to between −80 and +20 mV from a holding potential of −120 mV. FIG. 1(B) shows peak current density elicited by test pulses to various potentials and normalized to cell capacitance recorded during sequential superfusion of control solution (open squares) followed by 30 μM ranolazine (filled circles). FIG. 1(C) presents voltage dependence of activation measured during voltage steps to between −80 and +20 mV plotted together with voltage dependence of fast inactivation determined with 100 ms prepulses to between −140 and −10 mV (symbols are the same as defined in B). Pulse protocols are shown as panel insets and fit parameters are provided in Table 1. FIG. 1(D) shows the time dependent recovery from fast inactivation following an inactivating prepulse of 100 ms to −10 mV (symbols are the same as defined in. FIG. 1(B). Pulse protocols are shown as panel insets and fit parameters are provided in Table 1.

FIG. 2 illustrates how ranolazine preferentially inhibits Naᵥ1.1 A persistent current. Tonic inhibition of Naᵥ1.1 peak and persistent current measured using a 200 ms voltage step to −10 mV from a holding potential of −120 mV. Representative TTX-subtracted whole-cell sodium currents recorded for WT-Naᵥ1.1, FIG. 2(A), and R1648H, FIG. 2(B), during sequential superfusion of control solution (black trace) followed by 30 μM ranolazine (gray trace). The dashed line indicates zero current level. FIGS. 2(C) and 2(D) graphically display how ranolazine exhibits a concentration dependent tonic block of WT-Naᵥ1.1 and R1648H peak (open squares) and persistent (filled squares) currents. The peak and persistent current measured during ranolazine superfusion was normalized to the current measured in control solution. Fit parameters are provided in Table 2 in Example 1.

FIG. 3 presents data supporting the use-dependent block of Navᵥ1.1 by ranolazine. Naᵥ1.1 availability during repetitive stimulation was assessed with a depolarizing pulse train (−10 mV, 5 ms, 300 pulses, 10 Hz) from a holding potential of −120 mV. Representative whole-cell sodium currents recorded from WT-Naᵥ1.1 during sequential superfusion of control solution, as shown in FIG. 3(A) followed by 30 μM ranolazine, FIG. 3(B). Only the current traces from pulses 1, 30 and 300 are shown for clarity. FIGS. 3(C) and 3(D) graphically display how ranolazine exhibits concentration dependent and use-dependent block of WT-Naᵥ1.1 and R1648H peak currents (filled squares). Neither WT-Naᵥ1.1 nor R1648H exhibited use-dependent reduction in availability when exposed to drug-free control solution (open squares). Fit parameters are provided in Table 2 in Example 1.

FIG. 4 graphically illustrates the preferential block of persistent current by ranolazine. Tonic block of peak and persistent current measured using a 200 ms voltage step to −10 mV during application of 30 μM ranolazine for WT-Naᵥ1.1 and mutant Naᵥ1.1 channels. FIG. 4(A) graphically represents peak (filled bars) and persistent (open bars) current amplitudes were normalized to values recorded in drug-free control solution for each cell (n=5-7). FIG. 4(B) graphically represents persistent current expressed as a percentage of peak current recorded during the same voltage protocol for 30 μM ranolazine. Significant differences from WT-Naᵥ1.1 in drug-free solution are indicated by *(p<0.05) and ♦(p<0.01). FIG. 4(C) graphically represents use-dependent block of WT-Naᵥ1.1 and mutant channels during superfusion of 30 μM ranolazine (n=5-7). Neither WT-Naᵥ1.1 nor mutant Naᵥ1.1 channels exhibited use-dependent reduction in availability when exposed to drug-free control solution (filled bars). Significant differences from WT are indicated by *(p<0.05) and ♦(p<0.01).

FIG. 4 details how ranolazine inhibits ramp and use-dependent currents. FIG. 5(A) displays representative TTX-subtracted ramp currents measured during a 20 mV/s voltage ramp from a holding potential of −120 mV during sequential superfusion of control solution followed by 3 μM ranolazine. The dotted line indicates zero current. FIG. 5(B) graphically illustrates that R1648H conducted significantly more charge between −40 and 0 mV of the ramp, which was inhibited to the level of WT-Naᵥ1.1 by 3 μM ranolazine (n=9-10). FIG. 5(C) presents the data where WT-Naᵥ1.1 and R1648H availability was assessed during repetitive stimulation with a depolarizing pulse train (−10 mV, 5 ms, 300 pulses) at frequencies between 10 and 135 Hz during sequential superfusion of control solution followed ranolazine. Normalized peak current (pulse 300/pulse 1) was plotted versus frequency for each pulse train (n=9 and 8, respectively). FIG. 5(D) presents the curves showing inhibition of normalized peak current calculated as the ratio of channel availability during 3 μM ranolazine and control conditions. Significant differences between WT-Naᵥ1.1 and R1648H are indicated by *(p<0.05), ♦(p<0.01) and [symbol] (p<0.01).

### DETAILED DESCRIPTION OF THE INVENTION

### Definitions and General Parameters

As used in the present specification, the following words and phrases are generally intended to have the meanings as set forth below, except to the extent that the context in which they are used indicates otherwise.

“Optional” or “optionally” means that the subsequently described event or circumstance may or may not occur, and that the description includes instances where said event or circumstance occurs and instances in which it does not.

“Parenteral administration” is the systemic delivery of the therapeutic agent via injection to the patient.

The term “therapeutically effective amount” refers to that amount of a compound of Formula I that is sufficient to effect treatment, as defined below, when administered to a mammal in need of such treatment. The therapeutically effective amount will vary depending upon the specific activity of the therapeutic agent being used, the severity of the patient's disease state, and the age, physical condition, existence of other disease states, and nutritional status of the patient. Additionally, other medication the patient may be receiving will effect the determination of the therapeutically effective amount of the therapeutic agent to administer.

The term “treatment” or “treating” means any treatment of a disease in a mammal, including:
(i) preventing the disease, that is, causing the clinical symptoms of the disease not to develop;
(ii) inhibiting the disease, that is, arresting the development of clinical symptoms; and/or
(iii) relieving the disease, that is, causing the regression of clinical symptoms.

“Channelopathy” refers to a disease or condition that is associated with ion channel malformation.

Ranolazine is capable of forming acid and/or base salts by virtue of the presence of amino and/or carboxyl groups or groups similar thereto. The term “pharmaceutically acceptable salt” refers to salts that retain the biological effectiveness and properties of Ranolazine and which are not biologically or otherwise undesirable. Pharmaceutically acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl)amines, tri(substituted alkyl)amines, alkenyl amines, dialkenyl amines, trialkenyl amines, substituted alkenyl amines, di(substituted alkenyl)amines, tri(substituted alkenyl)amines, cycloalkyl amines, di(cycloalkyl)amines, tri(cycloalkyl)amines, substituted cycloalkyl amines, disubstituted cycloalkyl amine, trisubstituted cycloalkyl amines, cycloalkenyl amines, di(cycloalkenyl)amines, tri(cycloalkenyl)amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group.

Specific examples of suitable amines include, by way of example only, isopropylamine, trimethyl amine, diethyl amine, tri(iso-propyl)amine, tri(n-propyl)amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

Ranolazine, which is named N-(2,6-dimethylphenyl)-4-[2-hydroxy-3-(2-methoxyphenoxy)propyl]-1-piperazineacetamide {also known as 1-[3-(2-methoxyphenoxy)-2-hydroxypropyl]-4-[(2,6-dimethylphenyl)-aminocarbonylmethyl]-piperazine}, can be present as a racemic mixture, or an enantiomer thereof, or a mixture of enantiomers thereof, or a pharmaceutically acceptable salt thereof. Ranolazine can be prepared as described in U.S. Pat. No. 4,567,264, the specification of which is incorporated herein by reference.

“Immediate release” (“IR”) refers to formulations or dosage units that rapidly dissolve in vitro and are intended to be completely dissolved and absorbed in the stomach or upper gastrointestinal tract. Conventionally, such formulations release at least 90% of the active ingredient within 30 minutes of administration.

“Sustained release” (“SR”) refers to formulations or dosage units used herein that are slowly and continuously dissolved and absorbed in the stomach and gastrointestinal tract over a period of about six hours or more. Preferred sustained release formulations are those exhibiting plasma concentrations of Ranolazine suitable for no more than twice daily administration with two or less tablets per dosing as described below.

“Isomers” are different compounds that have the same molecular formula.

“Stereoisomers” are isomers that differ only in the way the atoms are arranged in space.

“Enantiomers” are a pair of stereoisomers that are non-superimposable minor images of each other. A 1:1 mixture of a pair of enantiomers is a “racemic” mixture. The term “(±)” is used to designate a racemic mixture where appropriate.

“Diastereoisomers” are stereoisomers that have at least two asymmetric atoms, but which are not minor-images of each other.

The absolute stereochemistry is specified according to the Cahn-Ingold-Prelog R-S system. When the compound is a pure enantiomer the stereochemistry at each chiral carbon may be specified by either R or S. Resolved compounds whose absolute configuration is unknown are designated (+) or (−) depending on the direction (dextro- or levorotary) which they rotate the plane of polarized light at the wavelength of the sodium D

### The Method of the Invention

The method of the invention is based on the surprising discovery that Ranolazine inhibits persistent Naᵥ1.1 current. Voltage-gated sodium channels are important targets for several widely used anti-epileptic drugs such as phenytoin and lamotrigine. These drugs act in part by stabilizing the inactivated state thereby reducing sodium channel availability and limiting the ability of neurons to fire repetitively. In addition to reducing sodium channel availability during repetitive neuronal activity, another potentially important effect of these drugs may be the suppression of persistent sodium current (Stafstrom C E (2007). Epilepsy Curr 7:15-22). Several types of neurons throughout the brain exhibit low amplitude persistent current resulting from incomplete closure of activated sodium channels. Although small, persistent sodium current can influence neuronal firing behavior substantially and may be critical to enabling spread of epileptic activity (Stafstrom, 2007).

The importance of persistent sodium current in the pathogenesis of epilepsy received additional attention when the functional consequences of neuronal sodium channel mutations discovered in various epilepsies were revealed. Several mutations in SCN1A associated with GEFS+ and other epilepsies exhibit increased persistent current sometimes as the predominant biophysical abnormality (Lossin et al. (2002). Neuron 34:877-884; Rhodes et al. (2004). Proc Natl Acad Sci USA 101:11147-11152; Kahlig K et al. (2006). J Neurosci 26:10958-10966; Kahlig et al (2008). Proc Natl Acad Sci USA 105:9799-9804; Spampanato et al. (2004). J Neurosci 24:10022-10034). These findings highlighted increased persistent current as a plausible pathophysiological factor in epileptogenesis and stimulated the idea that selective suppression of persistent current may offer a therapeutic strategy for rare familial epilepsies associated with mutations that promote this type of sodium channel dysfunction.

It has now been discovered that ranolazine, a drug approved for the treatment of chronic stable angina pectoris, is capable of selectively suppressing increased persistent current evoked by SCN1A mutations. It has now been determined that ranolazine exhibits 16-fold and 5-fold greater inhibition of persistent current as compared to tonic block and use-dependent block of peak current, respectively. This inhibition is concentration dependent with greatest selectivity in the low micromolar concentration range, which parallels the usual therapeutic plasma concentration of 2-10 μM (Sicouri et al. (2008). Heart Rhythm 5:1019-1026; Chaitman B R (2006). Circ 113:2462-2472).

While ranolazine does not have significant effects on current density, activation and voltage-dependence of inactivation, the compound does appear to slow recovery from inactivation which may indicate some degree of inactivated state stabilization. Ranolazine also exerts use-dependent block of WT and mutant Naᵥ1.1 providing further evidence of inactivation stabilization, but the concentrations required for these effects are much higher than the usual therapeutic plasma levels of the drug.

While not wishing to be bound by theory, the binding of ranolazine to Naᵥ1.1 and Naᵥ1.2 is believed to involve drug-receptor site interactions reported for other sodium isoforms. In a previous report investigating block of Naᵥ1.4 and Naᵥ1.7, Wang et al. determined that ranolazine selectively binds open states with minimal binding to either closed or inactivated states (Wang et al. (2008). Mol Pharmacol 73:940-948). Their study utilized voltage-train protocols with increasing step durations to correlate ranolazine use-dependent inhibition with the presentation of open conformations. The authors also reported a moderately rapid association rate (kon=8.2 μM-1 s-1) for Naᵥ1.4, which they suggested would allow drug binding only after channels respond normally to membrane depolarization. Unfortunately, to control current magnitude this study employed an inverse sodium gradient (65 mM external and 130 mM internal), and the resultant non-physiologic efflux of sodium ions may have affected drug binding kinetics, especially if ranolazine binds near the ion conduction pathway in open conformations. A second study by Rajamani et al. also investigated the state-dependent binding of ranolazine to Naᵥ1.7 and Naᵥ1.8 channels (Rajamani et al. (2008a). Channels 2:449-460).

The data presented in Example 1 combined with prior data highlight the diverse actions of ranolazine among sodium channel isoforms. Nevertheless, each study investigating the inhibition of sodium channels by ranolazine has reported preferential block of persistent current with a selectivity of between 9 and 17-fold (Wang et al. (2008); Fredj et al. (2006). Br J Pharmacol 148:16-24; Rajamani et al. (2009). Heart Rhythm 6:1625-1631).

Possible mechanisms of action for the persistent current block by ranolazine include, but are not limited to: 1) binding to open states and occluding the pore; 2) binding to open states and providing secondary inactivation stabilization; 3) binding to inactivated states to directly stabilization inactivation; or 4) a combination of each. Evidence for involvement of the intracellular local anesthetic binding site is supported by the observation that mutating the binding site in Naᵥ1.5 and Naᵥ1.4 reduces the efficacy of ranolazine (Wang et al. (2008); Fredj et al. (2006)).

At usual clinical dosages, ranolazine is well tolerated with a minority of patients experiencing mild adverse effects such as dizziness, nausea, headache and constipation (Nash et al. (2008). Lancet 372:1335-1341). Ranolazine also blocks the cardiac voltage-gated potassium channel HERG (Rajamani et al. (2008b). J Cardiovasc Pharmacol 51:581-589) and this accounts for the mild degree of QT interval prolongation observed in some subjects. As discussed in Example 1 below, it has now been determined that ranolazine is able to cross the blood-brain barrier, which may explain certain adverse effects such as dizziness and headache reported by subjects receiving the drug. Further, demonstration of ranolazine brain penetration supports the conclusion that this drug will exert an anti-epileptic effect in persons carrying certain sodium channel mutations such as those examined in Example 1.

In one embodiment of the invention, ranolazine is administered as a means to prevent epilepsy prophylaxis rather than in aborting active seizures based on the somewhat limited degree of use-dependent block exerted by the drug. Some degree of sodium channel use-dependent inhibition is likely important for an anticonvulsant effect and the therapeutic value of drugs selective for persistent current such as ranolazine might depend on the right balance of these two pharmacological actions. Thus, another embodiment of the invention is a method for treating CNS disorders comprising coadministration of a highly selective persistent current blocker with a more conventional anti-epileptic drug. Such a method will offer synergistic benefit to patients in need thereof.

### Utility Testing and Administration

### General Utility

The method of the invention is useful for treating CNS disorders including, but not limited to epilepsy and migraine. While not wishing to be bound by theory, it is believe that the ability of ranolazine to treat such CNS disorders is a result of its surprising capacity to act as an inhibitor of persistent Naᵥ1.1 and/or Naᵥ1.2 current in the brain.

### Pharmaceutical Compositions and Administration

Ranolazine is usually administered in the form of a pharmaceutical composition. This invention therefore provides pharmaceutical compositions that contain, as the active ingredient, ranolazine, or a pharmaceutically acceptable salt or ester thereof, and one or more pharmaceutically acceptable excipients, carriers, including inert solid diluents and fillers, diluents, including sterile aqueous solution and various organic solvents, solubilizers and adjuvants. Ranolazine may be administered alone or in combination with other therapeutic agents. Such compositions are prepared in a manner well known in the pharmaceutical art (see, e.g., Remington's Pharmaceutical Sciences, Mace Publishing Co., Philadelphia, Pa. 17^{th} Ed. (1985) and “Modern Pharmaceutics”, Marcel Dekker, Inc. 3^{rd} Ed. (G. S. Banker & C. T. Rhodes, Eds.).

Ranolazine may be administered in either single or multiple doses by any of the accepted modes of administration of agents having similar utilities, for example as described in those patents and patent applications incorporated by reference, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents.

Oral administration is the preferred route for administration of ranolazine. Administration may be via capsule or enteric coated tablets, or the like. In making the pharmaceutical compositions that include ranolazine, the active ingredient is usually diluted by an excipient and/or enclosed within such a carrier that can be in the form of a capsule, sachet, paper or other container. When the excipient serves as a diluent, it can be a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 50% by weight of the active compound, soft and hard gelatin capsules, sterile injectable solutions, and sterile packaged powders.

Ranolazine can also be formulated so as to provide quick, sustained or delayed release of the active ingredient after administration to the patient by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

Ranolazine is effective over a wide dosage range and is generally administered in a pharmaceutically effective amount. Typically, for oral administration, each dosage unit contains from 1 mg to 2 g of Ranolazine, more commonly from 1 to 700 mg, and for parenteral administration, from 1 to 700 mg of Ranolazine, more commonly about 2 to 200 mg. It will be understood, however, that the amount of Ranolazine actually administered will be determined by a physician, in the light of the relevant circumstances, including the condition to be treated, the chosen route of administration, the actual compound administered and its relative activity, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules.

The tablets or pills of the present invention may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

One mode for administration is parental, particularly by injection. The forms in which ranolazine may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection, but less preferred in the context of the present invention. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound of the invention in the required amount in the appropriate solvent with various other ingredients as enumerated above, as required, followed by filtration and sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The intravenous formulation of ranolazine is manufactured via an aseptic fill process as follows. In a suitable vessel, the required amount of Dextrose Monohydrate is dissolved in Water for Injection (WFI) at approximately 78% of the final batch weight. With continuous stirring, the required amount of ranolazine free base is added to the dextrose solution. To facilitate the dissolution of Ranolazine, the solution pH is adjusted to a target of 3.88-3.92 with 0.1N or 1N Hydrochloric Acid solution. Additionally, 0.1N HCl or 1.0N NaOH may be utilized to make the final adjustment of solution to the target pH of 3.88-3.92. After ranolazine is dissolved, the batch is adjusted to the final weight with WFI. Upon confirmation that the in-process specifications have been met, the Ranolazine bulk solution is sterilized by sterile filtration through two 0.2 μm sterile filters. Subsequently, the sterile ranolazine bulk solution is aseptically filled into sterile glass vials and aseptically stoppered with sterile stoppers. The stoppered vials are then sealed with clean flip-top aluminum seals.

The compositions are preferably formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet, capsule, ampule). It will be understood, however, that the amount of ranolazine actually administered will be determined by a physician, in the light of the relevant circumstances, including the condition to be treated, the chosen route of administration, the actual compound administered and its relative activity, the age, weight, and response of the individual patient, the severity of the patient's symptoms, and the like.

In one embodiment, the ranolazine is formulated so as to provide quick, sustained or delayed release of the active ingredient after administration to the patient, especially sustained release formulations. Unless otherwise stated, the ranolazine plasma concentrations used in the specification and examples refer to ranolazine free base.

The preferred sustained release formulations of this invention are preferably in the form of a compressed tablet comprising an intimate mixture of compound and a partially neutralized pH-dependent binder that controls the rate of dissolution in aqueous media across the range of pH in the stomach (typically approximately 2) and in the intestine (typically approximately about 5.5). An example of a sustained release formulation is disclosed in U.S. Pat. Nos. 6,303,607; 6,479,496; 6,369,062; and 6,525,057, the complete disclosures of which are hereby incorporated by reference.

### Combination Therapy

Patients being treated for CNS disorders such as epilepsy often benefit from treatment with more than one therapeutic agent. Commonly used anticonvulsant medications include carbamazepine, phenobarbital, phenytoin, and valproic acid. Other commonly use antiepileptic drugs include, but are not limited to, gabapentin, lamotrigine, topiramate, ethosuximide, clonazepam, and acetazolamide.

The co-administration of ranolazine with a therapeutically effective amount of at least one antiepileptic medication allows enhancement in the standard of care therapy the patient is currently receiving. Accordingly, one aspect of the invention provides a method for treating a CNS disorder comprising administration of a therapeutically effective amount of ranolazine and a therapeutically effective amount of at least one antiepileptic medication to a mammal in need thereof.

The methods of combination therapy include coadministration of a single formulation containing the ranolazine and therapeutic agent or agents, essentially contemporaneous administration of more than one formulation comprising the ranolazine and therapeutic agent or agents, and consecutive administration of ranolazine and therapeutic agent or agents, in any order, wherein preferably there is a time period where the ranolazine and therapeutic agent or agents simultaneously exert their therapeutic affect. Preferably the ranolazine is administered in an oral dose as described herein.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Material and Methods

Expression of Human Naᵥ1.1 cDNA

All wild-type (WT) and mutant constructs have been studied previously by our laboratory (Kahlig, 2008; Lossin, 2002; Rhodes, 2004) and cDNA expression was performed as previously described (Kahlig, 2008). Briefly, expression of Naᵥ1.1 was achieved by transient transfection using Qiagen Superfect reagent (5.5 μg of DNA was transfected at a plasmid mass ratio of 10:1:1 for a₁:β₁:β₂). The human β₁ and β₂ cDNAs were cloned into plasmids containing the marker genes DsRed (DsRed-IRES2-hβ₁) or EGFP (EGFP-IRES2-hβ₂) along with an internal ribosome entry site (IRES). Unless otherwise noted, all reagents were purchased from Sigma-Aldrich (St Louis, Mo., U.S.A.).

### Electrophysiology

Whole-cell voltage-clamp recordings were used to measure the biophysical properties of WT and mutant Naᵥ1.1 channels, as described previously (Kahlig, 2008). Briefly, the pipette solution consisted of (in mM) 110 CsF, 10 NaF, 20 CsCl, 2 EGTA, 10 HEPES, with a pH of 7.35 and osmolarity of 300 mOsmol/kg. The bath (control) solution contained in (mM): 145 NaCl, 4 KCl, 1.8 CaCl₂, 1 MgCl₂, 10 dextrose, 10 HEPES, with a pH of 7.35 and osmolarity of 310 mOsmol/kg. Cells were allowed to stabilize for 10 min after establishment of the whole-cell configuration before current was measured. Series resistance was compensated 90% to assure that the command potential was reached within microseconds with a voltage error <2 mV. Leak currents were subtracted by using an online P/4 procedure and all currents were low-pass Bessel filtered at 5 kHz and digitized at 50 kHz. For clarity, representative ramp currents were low pass filtered off-line at 50 Hz.

Specific voltage-clamp protocols assessing channel activation, fast inactivation and availability during repetitive stimulation were used as depicted as figure insets. Whole-cell conductance was calculated from the peak current amplitude by G_{Na}=I_{Na}/(V-E_{Na}) and normalized to the maximum conductance between −80 and +20 mV. Conductance-voltage and steady-state channel availability curves were fit with Boltzmann functions to determine the voltage for half-maximal activation/inactivation (V_{1/2}) and a slope factor (k). Time-dependent entry into and recovery from inactivation were evaluated by fitting the peak current recovery with the two exponential function, I/Iₘₐₓ=A_{f}×[1−exp(−t/τ_{f})]+Aₛ×[1−exp(−t/τₛ)], where τ_{f} and τₛ denote time constants (fast and slow components, respectively), A_{f} and Aₛ represent the fast and slow fractional amplitudes.

For use-dependent studies, cells were stimulated with depolarizing pulse trains (−10 mV, 5 ms, 300 pulses, 10 Hz) from a holding potential of −120 mV. Currents were then normalized to the peak current recorded in response to the first pulse in each frequency train. For tonic block studies, peak and persistent current were evaluated in response to a 200 ms depolarization to −10 mV (0.2 Hz) following digital subtraction of currents recorded in the presence and absence of 0.5 μM tetrodotoxin (TTX). Persistent current was calculated during the final 10 ms of the 200 ms step. Data analysis was performed using Clampfit 9.2 (Axon Instruments, Union City, Calif., U.S.A), Excel 2002 (Microsoft, Seattle, Wash., U.S.A.), and OriginPro 7.0 (OriginLab, Northampton, Mass., U.S.A) software. Results are presented as mean±SEM. Unless otherwise noted, statistical comparisons were made using one-way ANOVA followed by a Tukey post-hoc test in reference to WT-Naᵥ1.1.

In vitro Pharmacology

A stock solution of 20 mM ranolazine (Gilead, Foster City, Calif.) was prepared in 0.1 M HCl. A fresh dilution of ranolazine in the bath solution was prepared every experimental day and the pH was readjusted to 7.35. Direct application of the perfusion solution to the clamped cell was achieved using the Perfusion Pencil system (Automate, Berkeley, Calif.). Direct cell perfusion was driven by gravity at a flow rate of 350 μL/min using a 250 micron tip. This system sequesters the clamped cell within a perfusion stream and enables complete solution exchange within 1 second. The clamped cell was perfused continuously starting immediately after establishing the whole-cell configuration. Control currents were measured during control solution perfusion.

Ranolazine containing solutions were perfused for three minutes prior to current recordings to allow equilibrium (tonic) drug block. Tonic block of peak and persistent currents were measured from this steady-state condition. Three sequential current traces were averaged to obtain a mean current for each recording condition (control, ranolazine and TTX). The mean current traces were utilized for offline subtraction and analysis. Use-dependent block of peak current was measured during pulse number 300 of the pulse train, (−10 mV, 5 ms, 300 pulses, 10 Hz) from a holding potential of −120 mV. Two sequential pulse train stimulations were averaged to obtain mean current traces for each recording condition, which were then used for offline subtraction and analysis. Block of ramp current was assessed by voltage ramps to +20 mV from a holding potential of −120 mV at a rate of 20 mV/s stimulated every 30 s. To minimize time-dependent current drift, only one trace recorded during control, ranolazine or TTX superfusion was analyzed. TTX was applied in the presence of ranolazine. Concentration inhibition curves were fit with the Hill equation: I/Iₘₐₓ=1/[1+10̂(logIC₅₀−I)*k], where IC₅₀ is the concentration that produces half inhibition and k is the Hillslope factor.

In vivo Pharmacology

Jugular vein cannulated male Sprague Dawley rats (250-350 g, Charles River Laboratories, Hollister, Calif.) were used to study brain penetration of ranolazine in vivo. Animal use was approved by the Institutional Animal Care and Use Committee, Gilead Sciences. Three rats per group were infused intravenously with ranolazine in saline at 85.5 μg/kg/min. After 1, 2.5 or 5 h animals were sacrificed for plasma and brain collection, and ranolazine concentrations were measured by liquid chromatography coupled with tandem mass spectrometry (LC-MS/MS). Brain tissue was homogenated in 1% 2N HCl acidified 5% sodium fluoride (final homogenate was diluted 3-fold). Plasma and brain homogenate samples (50 μl) were precipitated along with deuterated D3-ranolazine as an internal standard, vortexed and centrifuged. The supernatant (50 μL) was transferred and diluted with water (450 μl) prior to injection (10 μl). High performance liquid chromatography was performed using a Shimadzu LC-10AD liquid chromatograph and a Luna C18(2), 3 μm, 20×2.0 mm column with a mobile phase consisting of water containing 0.1% formic acid (solution A) and acetonitrile (solution B) carried out under isocratic conditions (75% solution A, 25% solution B; flow rate 0.300 ml/min). Mass spectrometric analyses were performed using an API3000 mass spectrometer (Applied Biosystems, Foster City, Calif.) operating in positive ion mode with MRM transition 428.1>98. Brain-to-plasma ranolazine ratios were calculated for each sample as ng ranolazine/g brain divided by ng ranolazine/ml plasma.

### Results

It has now been demonstrated that ranolazine has the ability to inhibit WT-Naᵥ1.1 and a panel of Naᵥ1.1 mutant channels associated with the epilepsy and migraine syndromes GEFS+, SMEI and FHM3 demonstrating the ability of ranolazine to preferentially block the abnormal increased persistent current carried by these mutant channels.

### Ranolazine Effects on WT-Naᵥ1.1 Activation and Inactivation

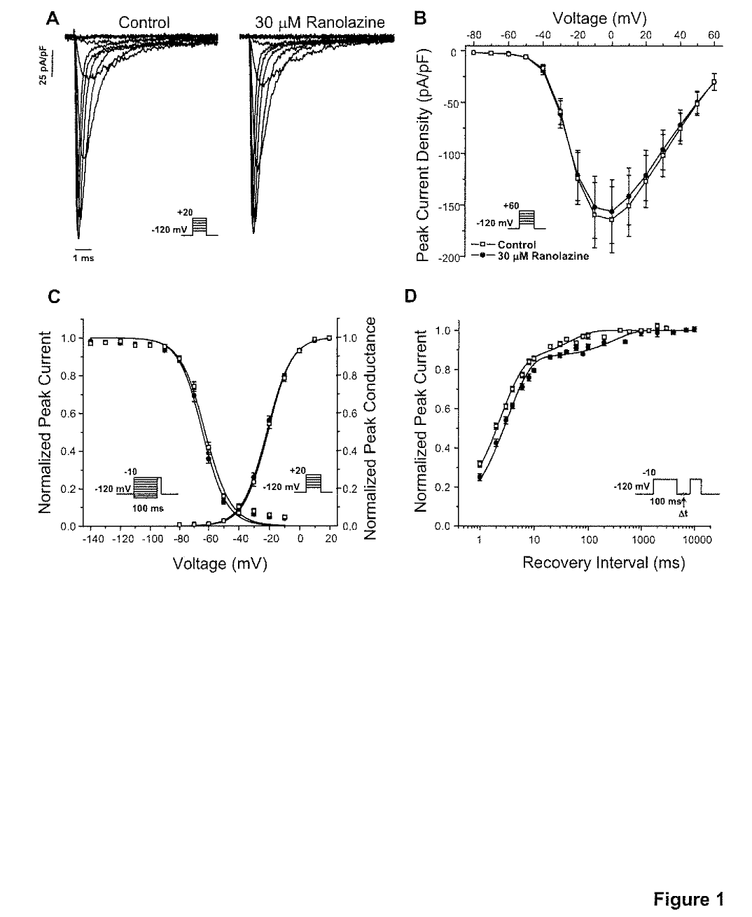

The ability of ranolazine to alter the activation and inactivation properties of WT-Naᵥ1.1 expressed heterologously in tsA201 cells was determined. FIG. 1(A) illustrates representative whole-cell sodium currents recorded from a cell expressing WT-Naᵥ1.1 in control solution (drug-free) and the same cell during superfusion with 30 μM ranolazine. Application of the drug had no overt effects on WT-Naᵥ1.1 function even at this high concentration. Similarly, there was no significant effect of the drug on peak current density recorded during sequential application of control solution and 30 μM ranolazine (FIG. 1(B)). Furthermore, 30 μM ranolazine did not significantly shift the voltage-dependence of WT-Naᵥ1.1 activation or inactivation (FIG. 1(C), Table 1). These results indicate that ranolazine does not interfere with activation of the channel. However, 30 μM ranolazine did cause a slight but significant slowing of recovery from inactivation (FIG. 1(D), Table 1) consistent with increased stability of the inactivated state. These results indicate that 30 μM ranolazine has minimal effects on WT-Naᵥ1.1 function.

TABLE 1
Biophysical Parameters for WT-Naᵥ1.1 Activation and Fast Inactivation

| | Activation | | | Inactivation | | | Recovery from Inactivation^{§} | | |
|---|---|---|---|---|---|---|---|---|---|
| | V_{1/2} (mV) | k (mV) | n | V_{1/2} (mV) | k (mV) | n | T_{f} (ms) | k (mV) | n |
| Control | −20.9 ± 0.9 | 7.7 + 0.2 | 10 | −63.3 ± 0.8 | −8.6 ± 0.6 | 10 | 2.2 ± 0.2 | 63.5 ± 12.1 | 10 |
| | | | | | | | [82 ± 4%] | [18 + 4%] | |
| 30 μM | −21.6 ± 0.8 | 8.0 ± 0.2 | 10 | −64.2 ± 0.8 | −8.0 ± 0.5 | 10 | 3.2 ± 0.2** | 412.4 ± 86.1** | 10 |
| Ranolazine | | | | | | | [85 ± 1%] | [15 ± 1%] | |

^{§}Values in brackets represent fraction amplitudes.
Values significantly different from Control are indicated as follows
*p < 0.05,
**p < 0.01.

### Preferential Ranolazine Block of Persistent Current

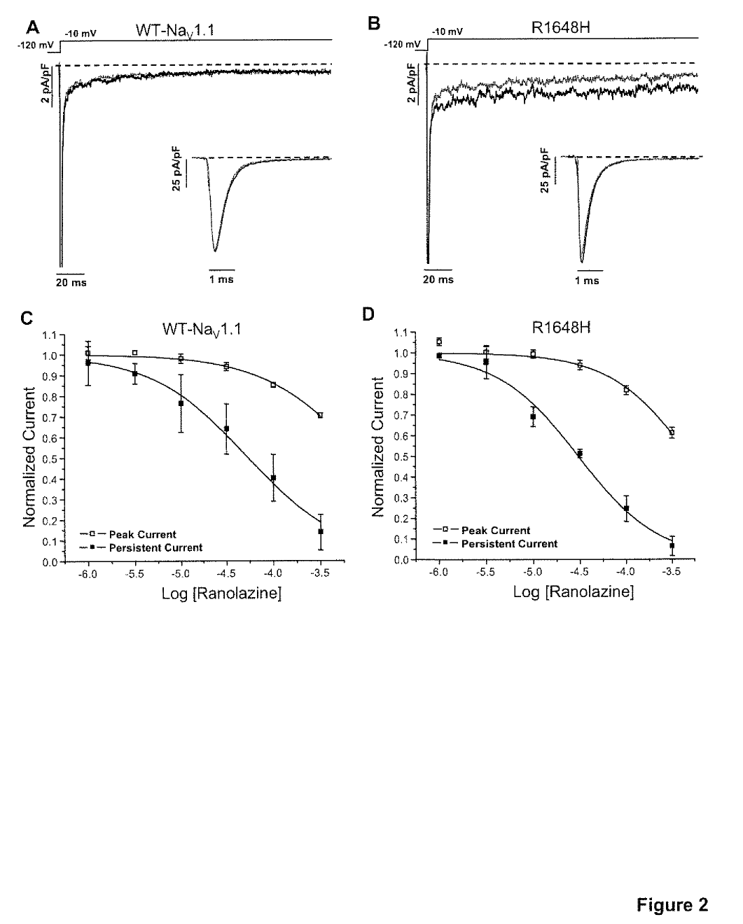

We examined the concentration dependent tonic inhibition of peak and persistent current carried by WT-Naᵥ1.1 and a mutant Naᵥ1.1 (R1648H) associated with GEFS+ that we previously demonstrated to exhibit significantly increased persistent current as the only apparent biophysical defect (Lossin et al., 2002; Kahlig et al., 2006). FIG. 2(A) illustrates whole-cell sodium currents recorded from WT-Naᵥ1.1 during sequential application of control solution (black trace) followed by 30 μM ranolazine (gray trace). Tonic block of WT-Naᵥ1.1 peak current was minimal as illustrated by the figure inset where the data were plotted on an expanded time scale. In FIG. 2(B), which illustrates the same experimental sequence for R1648H, persistent current was substantially reduced during superfusion of ranolazine as compared to the drug-free condition. As observed for WT-Naᵥ1.1, 30 μM ranolazine exerted minimal tonic block of R1648H peak current (FIG. 2(B) inset).

Ranolazine exhibited greater degrees of tonic inhibition of persistent current as compared with peak current for both WT-Naᵥ1.1 and R1648H (FIGS. 2(C) and 2(D), respectively). Fits of concentration-inhibition curves with the Hill equation provided IC₅₀ values of 871 μM for WT-Naᵥ1.1 and 490 μM for R1648H for tonic peak current block (Table 2), whereas ranolazine block of persistent current carried by WT-Naᵥ1.1 and R1648H exhibited IC₅₀ values of 53.7 μM and 30.2 μM, respectively. These results demonstrate that ranolazine has approximately 16-fold selectivity for tonic block of persistent current carried by either WT-Naᵥ1.1 or R1648H.

TABLE 2
Tonic and use-dependent block of WT-Naᵥ1.1 and R1648H

| | Tonic Block of | | Tonic Block of | | Use-Dependent Block of | |
|---|---|---|---|---|---|---|
| | Peak Current | | Persistent Current | | Peak Current | |
| | LogIC₅₀ | k | LogIC₅₀ | k | LogIC₅₀ | k |
| WT-Naᵥ1.1 | −3.06 ± 0.13 | −0.84 ± 0.15 | −4.27 ± 0.04 | −0.83 ± 0.06 | −3.71 ± 0.02 | −0.84 ± 0.05 |
| | (871 uM) | | (53.7 uM) | | (195 uM) | |
| R1648H | −3.31 ± 0.09 | −1.00 ± 0.19 | −4.52 ± 0.04 | −1.00 ± 0.09 | −3.86 ± 0.02 | −0.88 ± 0.03 |
| | (490 uM) | | (30.2 uM) | | (138 uM) | |

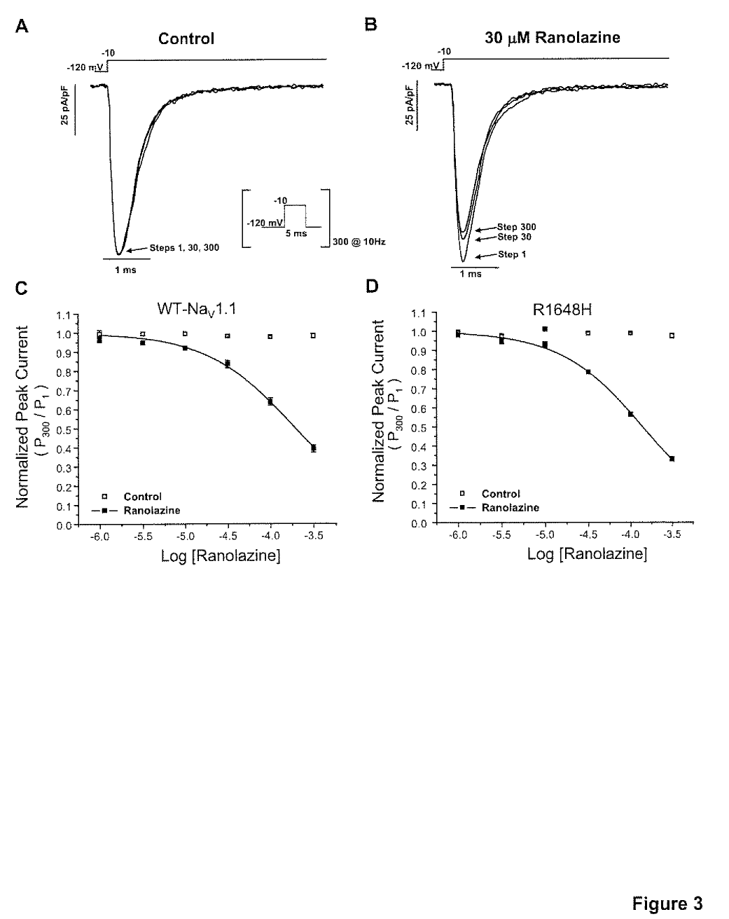

We also assessed use-dependent block of WT-Naᵥ1.1 and R1648H by ranolazine. FIG. 3A illustrates the whole-cell sodium currents recorded from WT-Naᵥ1.1 in response to a repetitive depolarization protocol (5 ms, −10 mV, 300 pulses, 10 Hz) during superfusion of control solution. In the drug-free control condition, the availability of WT-Naᵥ1.1 is unchanged during repetitive depolarization. In contrast, application of 30 μM ranolazine to the same cell caused a reduction in peak current during repetitive pulsing consistent with use-dependent block of the channel (FIG. 3(B)). The concentration dependence of ranolazine use-dependent block of WT-Naᵥ1.1 and R1648H was characterized by IC₅₀ values of 195 μM and 138 μM, respectively (FIGS. 3(C) and 3(D), Table 2). These results demonstrated that ranolazine was 3.6-fold and 4.6-fold more potent at inhibiting persistent current carried by WT-Naᵥ1.1 and R1648H, respectively, as compared to use-dependent block of peak current.

### Ranolazine Block of Mutant Naᵥ1.1 Channels

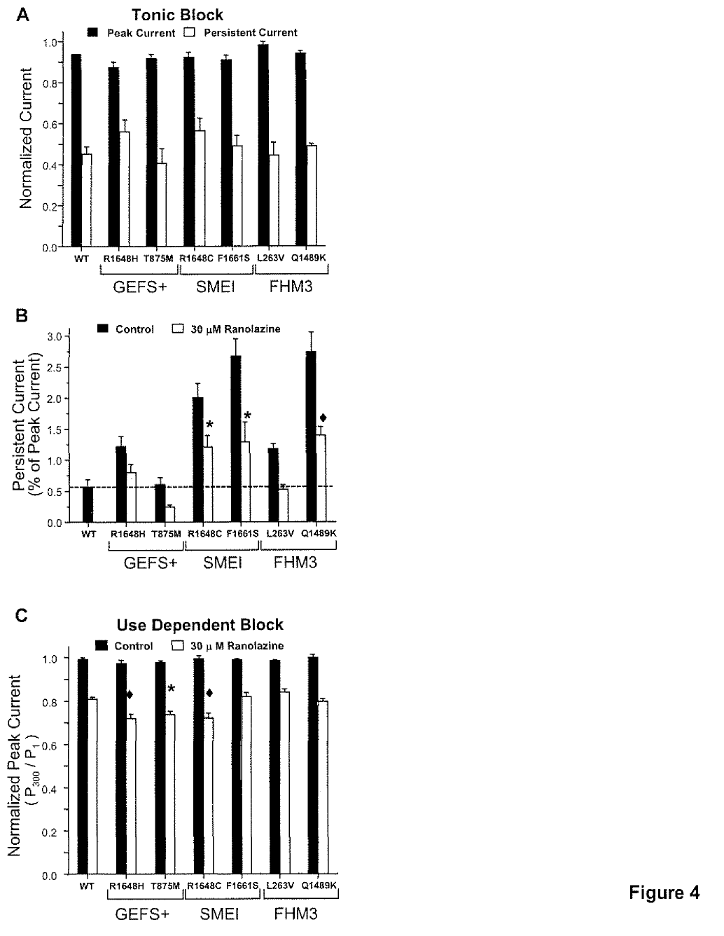

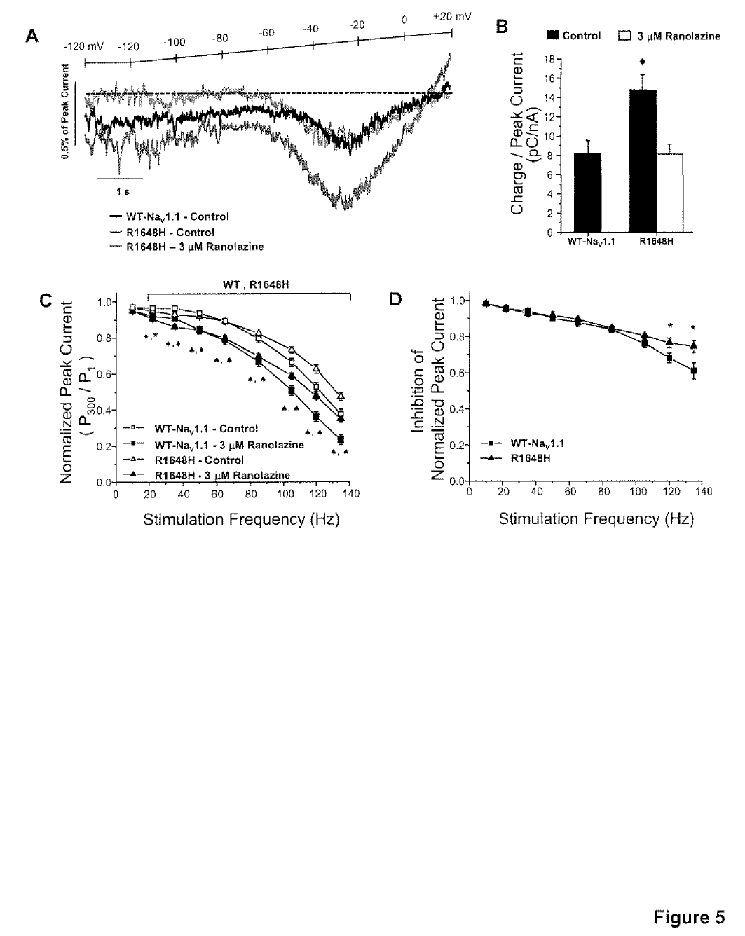

We compared the degree of ranolazine block among six Naᵥ1.1 mutant channels representing three clinical syndromes: GEFS+ (R1648H, T875M), SMEI (R1648C, F1661S) and FHM3 (L263V, Q1489K). FIG. 4(A) illustrates tonic block of peak and persistent current by 30 μM ranolazine for this panel of mutant channels normalized to current amplitudes recorded in drug-free control solution. For all mutants, we observed a much greater degree of ranolazine block of persistent current as compared to peak current. We also assessed the ability of ranolazine to reduce the magnitude of persistent current exhibited by mutant channels to the level conducted by WT-Naᵥ1.1. In FIG. 4(B), persistent current was expressed as a percent of peak current and was not normalized to the drug-free condition. In general, the level of persistent current carried by mutant channels was reduced by approximately 50% (range 44-60%), but for some mutants (R1648H, T875M, L263V) the level in the presence of ranolazine was not significantly different from WT-Naᵥ1.1 channels in the absence of drug.

We also assessed use-dependent block of mutant Naᵥ1.1 peak currents by ranolazine. FIG. 4(C) illustrates use-dependent block of peak current for WT-Naᵥ1.1 and mutant channels by 30 μM ranolazine. Neither WT-Naᵥ1.1 nor any mutant channel exhibited significant loss of channel availability in control solution by the 300^{th} pulse, but there was significant loss of channel availability during ranolazine application for both WT-Naᵥ1.1 and mutant channels. However, the mutants R1648H, T875M and R1648C exhibited a significantly greater reduction in channel availability in the presence of 30 μM ranolazine as compared to WT-Naᵥ1.1.

By dividing the degree of persistent current block by the extent of use-dependent block of peak current, we calculated a selectivity index for the effect of ranolazine on mutant Naᵥ1.1 channels. Ranolazine exhibited the most selective block of persistent current on L263V and F1661S, and least selective block on R1648H and R1648C channels with an overall rank order of L263V>F1661S>Q1489K>T875M>R1648H=R1648C. These relationships may help predict molecular subsets of Naᵥ1.1 mutations that might be more amenable to selective suppression of increased persistent current.

### Brain Penetration of Ranolazine

The ability of ranolazine to cross the blood brain barrier has not been reported previously. We measured the degree of brain penetration of ranolazine in rats following continuous intravenous infusion of the drug (85.5 μg/kg/min) for 1, 2.5 and 5 h. Ranolazine exhibited significant brain penetration at all time points peaking after 5 hours at 470 ng ranolazine/g brain (approximately 1.1 μM, Table 3). Throughout the time course studied, the mean brain levels of ranolazine were approximately one third of the corresponding plasma levels. Given that the therapeutic plasma concentration of ranolazine is 2-10 μM, brain concentrations up to 3.3 μM should be feasible.

TABLE 3
Ranolazine brain penetration

| | | | Brain/ |
|---|---|---|---|
| Time (h) | Brain (ng/g) | Plasma (ng/mL) | Plasma (%) |
| 1 | 298 ± 88.6 (0.70 uM) | 777 ± 255 (1.82 uM) | 38 |
| 2.5 | 446 ± 302 (1.04 uM) | 1180 ± 456 (2.76 uM) | 38 |
| 5 | 470 ± 300 (1.10 uM) | 1590 ± 488 (3.72 uM) | 30 |

### Suppression of Persistent Current by Therapeutic Ranolazine Concentration

We next examined the ability of 3 μM ranolazine, an achievable brain concentration, to suppress R1648H activation during slow depolarizing voltage ramps, a phenomenon attributed to increased persistent current. FIG. 5(A) shows representative inward currents produced in response to a slow depolarizing voltage ramp. R164811 cells exhibited an increased depolarizing current (compared to WT; medium gray versus black traces) that was blocked by 3 μM ranolazine (light gray trace). The average inward charge (pC) was calculated for multiple cells as the area under the current trace between −40 and 0 mV and normalized to the corresponding peak current (nA) generated by a voltage step to −10 mV to account for variation in channel expression. FIG. 5(B) demonstrates that sequential superfusion of control solution followed by 3 μM ranolazine reduced the charge conducted by R1648H to the level observed in cells expressing WT channels recorded in the absence of drug.

Finally, we assessed use-dependent block of WT and mutant Naᵥ1.1 by 3 μM ranolazine. FIG. 5(C) illustrates use-dependent block of WT-Naᵥ1.1 and R1648H channels at pulsing frequencies between 10 and 135 Hz. In control solution, both WT and R1648H exhibited an expected degree of frequency-dependent loss of channel availability, while application of 3 μM ranolazine exaggerated loss of availability at all frequencies greater than 22 Hz. These results are consistent with significant use-dependent block by 3 μM ranolazine. FIG. 5D shows that 3 μM ranolazine produced a similar degree of block of WT and R1648H channels up to 100 Hz.

### Example 2

### Material and Methods

Expression of Human Naᵥ1.2 cDNA

Wild-type (WT) cDNA stably transfected in Chinese hamster ovary (CHO) cells is used to record Na+ currents. Unless otherwise noted, all reagents are purchased from Sigma-Aldrich (St Louis, Mo., U.S.A.).

### Electrophysiology

Whole-cell voltage-clamp recordings are used to measure the biophysical properties of WT. Briefly, the pipette solution consists of (in mM) 110 CsF, 10 NaF, 20 CsCl, 2 EGTA, 10 HEPES, with a pH of 7.35 and osmolarity of 300 mOsmol/kg. The bath (control) solution contains in (mM): 145 NaCl, 4 KCl, 1.8 CaCl2, 1 MgCl2, 10 dextrose, 10 HEPES, with a pH of 7.35 and osmolarity of 310 mOsmol/kg. Cells are allowed to stabilize for 10 min after establishment of the whole-cell configuration before current is measured. Series resistance is compensated 90% to assure that the command potential is reached within microseconds with a voltage error <2 mV. Leak currents are subtracted by using an online P/4 procedure and all currents are low-pass Bessel filtered at 5 kHz and digitized at 50 kHz.

For clarity, representative ramp currents are low pass filtered off-line at 50 Hz. Specific voltage-clamp protocols assessing channel activation, fast inactivation and availability during repetitive stimulation are used. Results are presented as mean±SEM, and unless otherwise noted, statistical comparisons are made using one-way ANOVA.

Tonic block of peak current is measured. The mean current traces are utilized for offline subtraction and analysis. Use-dependent block of peak current is measured during pulse number 300 of a pulse train (−10 mV, 5 ms, 300 pulses) at frequencies between 10 and 135 Hz from a holding potential of −120 mV. Two sequential pulse train stimulations are averaged to obtain mean current traces for each recording condition, which are then used for offline subtraction and analysis.

Specific voltage-clamp protocols assessing channel activation, fast inactivation and availability during repetitive stimulation are used. Whole-cell conductance is calculated from the peak current amplitude by G_{Na}=I_{Na}/(V−E_{Na}) and normalized to the maximum conductance between −80 and +20 mV. Conductance-voltage and steady-state channel availability curves are fit with Boltzmann functions to determine the voltage for half-maximal activation/inactivation (V_{1/2}) and a slope factor (k). Time-dependent entry into and recovery from inactivation are evaluated by fitting the peak current recovery with the two exponential function, I/Iₘₐₓ=A_{f}×[1−exp(−t/τ_{f})]+Aₛ×[1−exp(−t/τₛ)], where τ_{f} and τₛ denote time constants (fast and slow components, respectively), A_{f} and Aₛ represent the fast and slow fractional amplitudes.

For use-dependent studies, cells are stimulated with depolarizing pulse trains (−10 mV, 5 ms, 300 pulses, 10 Hz) from a holding potential of −120 mV. Currents are then normalized to the peak current recorded in response to the first pulse in each frequency train. For tonic block studies, peak and persistent current are evaluated in response to a 200 ms depolarization to −10 mV (0.2 Hz) following digital subtraction of currents recorded in the presence and absence of 0.5 μM tetrodotoxin (TTX). Persistent current is calculated during the final 10 ms of the 200 ms step. Data analysis is performed using Clampfit 9.2 (Axon Instruments, Union City, Calif., U.S.A), Excel 2002 (Microsoft, Seattle, Wash., U.S.A.), and OriginPro 7.0 (OriginLab, Northampton, Mass., U.S.A) software. Results are presented as mean±SEM. Unless otherwise noted, statistical comparisons are made using one-way ANOVA followed by a Tukey post-hoc test in reference to WT-Naᵥ1.2.

In vitro Pharmacology

A stock solution of 20 mM ranolazine (Gilead, Foster City, Calif.) is prepared in 0.1 M HCl. A fresh dilution of ranolazine in the bath solution was prepared every experimental day and the pH is readjusted to 7.35. Direct application of the perfusion solution to the clamped cell is achieved using the Perfusion Pencil system (Automate, Berkeley, Calif.). Direct cell perfusion is driven by gravity at a flow rate of 350 μL/min using a 250 micron tip. This system sequesters the clamped cell within a perfusion stream and enables complete solution exchange within 1 second. The clamped cell is perfused continuously starting immediately after establishing the whole-cell configuration. Control currents are measured during control solution perfusion.

Ranolazine containing solutions are perfused for three minutes prior to current recordings to allow equilibrium (tonic) drug block. Tonic block of peak and persistent currents are measured from this steady-state condition. Three sequential current traces are averaged to obtain a mean current for each recording condition (control, ranolazine and TTX). The mean current traces are utilized for offline subtraction and analysis. Use-dependent block of peak current is measured during pulse number 300 of the pulse train, (−10 mV, 5 ms, 300 pulses, 10 Hz) from a holding potential of −120 mV. Two sequential pulse train stimulations are averaged to obtain mean current traces for each recording condition, which are then used for offline subtraction and analysis. Block of ramp current is assessed by voltage ramps to +20 mV from a holding potential of −120 mV at a rate of 20 mV/s stimulated every 30 s. To minimize time-dependent current drift, only one trace recorded during control, ranolazine or TTX superfusion is analyzed. TTX is applied in the presence of ranolazine. Concentration inhibition curves are fit with the Hill equation: I/Iₘₐₓ=1/[1+10̂(logIC₅₀−I)*k], where IC₅₀ is the concentration that produces half inhibition and k is the Hill slope factor.

### Results

It is thus demonstrated that ranolazine has the ability to inhibit WT-Naᵥ1.2 demonstrating the ability of ranolazine to preferentially block an abnormal increased persistent current carried by this channel.

We claim:

1. A method for treating central nervous system disorders comprising administration of a therapeutically effective amount of ranolazine to a mammal in need thereof.

2. The method of claim 1 wherein the central nervous system disorder is migraine or epilepsy.

3. The method of claim 1 wherein the central nervous system disorder is associated with SCN1A mutation.

4. The method of claim 3, wherein the central nervous system disorder is associated with a SCN1A mutation.

5. The method of claim 3, wherein the central nervous system disorder is selected from the group consisting of generalized epilepsy with febrile seizures plus (GEFS+) type 2, severe myoclonic epilepsy of infancy (SMEI), familial hemiplegic migraine type 3 (FHM3), generalized epilepsy with febrile seizures plus (GEFS+) type 1.

6. The method of claim 1 wherein ranolazine is in the form of a pharmaceutically acceptable salt.

7. The method of claim 6 wherein the pharmaceutically acceptable salt is the dihydrochloride salt.

8. The method of claim 1 wherein ranolazine is in the form of the free base.

9. A method for treating central nervous system disorders comprising administration of a therapeutically effective amount of ranolazine and a therapeutically effective amount of at least one antiepileptic medication to a mammal in need thereof.

10. The method of claim 9, wherein the antiepileptic medication is selected from the group consisting of carbamazepine, phenobarbital, phenytoin, valproic acid, gabapentin, lamotrigine, topiramate, ethosuximide, clonazepam, and acetazolamide.

11. The method of claim 10, wherein the ranolazine and the antiepileptic medication are administered as separate dosage forms.

12. The method of claim 10, wherein ranolazine and the antiepileptic medication are administered as a single dosage form.

13. The method of claim 10, wherein the ranolazine and the antiepileptic medication are administered as separate dosage forms.

14. The method of claim 10, wherein ranolazine and the antiepileptic medication are administered as a single dosage form.

15. A pharmaceutical formulation comprising a therapeutically effective amount of ranolazine, a therapeutically effective amount at least one c antiepileptic medication, and at least one pharmaceutically acceptable carrier.
